(19) United States
(12) Patent Application Publication
(10) Pub. No.: US 2015/0245897 A1
(43) Pub. Date: Sep. 3, 2015

(54) Animal Leg Protector

(71) Applicants: Dariush Hasebi, Cottonwood Heights, UT (US); Weiwen Hasebi, Cottonwood Heights, UT (US)

(72) Inventors: Dariush Hasebi, Cottonwood Heights, UT (US); Weiwen Hasebi, Cottonwood Heights, UT (US)

(21) Appl. No.: 14/485,710

(22) Filed: Sep. 13, 2014

Related U.S. Application Data
(60) Provisional application No. 61/878,637, filed on Sep. 17, 2013.

(51) Int. Cl. A61D 9/00; A61F 15/00; A01K 13/00

(52) U.S. Cl. CPC A61D 9/00; A01K 13/007; A61F 15/006

(57) ABSTRACT

The present invention relates to apparatus for protecting wounds on the animal's leg or paw. The apparatus comprises a sleeve sized and shaped to loosely enclose the animal's leg and paw, as well as two straps. The sleeve includes a closed bottom paw area sized and shaped to contain the animal's paw, and a leg area connected to the top of the paw area sized and shaped to contain the animal's leg. The sleeve further includes an armpit patch made of flexible material located at the upper front or rear portion of the sleeve to adjust comfortably to the animal's armpit.

### REFERENCE TO RELATED APPLICATIONS

This patent application claims the benefit of U.S. Provisional Application No. 61/878,637 filed on Sep. 17th, 2013, the disclosure of which is incorporated herein by reference in its entirety.

### BACKGROUND OF THE INVENTION

1. Field of the Invention

This invention relates to an animal leg protector and, more specifically to a sleeve made of cloth or other flexible material that can enclose the animal's leg and paw to protect any sore, wound or other injury on the animal's leg and paw.

2. Description of Related Art

Frequently, when an animal has a wound, sore or injury to its leg, or has stitches, the animal will attempt to lick the wound. This presents the danger of infection and tends to slow healing and recovery. This is currently prevented by the use of a cone on the animal's neck during the healing process. However, cones tend to be bulky and uncomfortable and make it difficult for the animal to turn around inside a pen or kennel.

Apparatus for providing protection to animals' wound is known in the prior art. More specifically, by way of example, U.S. Pat. No. 5,076,043 to Buttler discloses a device having two sleeves that envelope the animal's legs. A pair of straps is provided to connect the straps together and secure them to the animal. The sleeves are made of a leg area having an internal and external fabric layer with a padding material placed therebetween.

U.S. Pat. No. 5,341,765 to McComb discloses a protective garment formed a single piece of material that forms a strap extending across the animal's back and has ends formed into sleeves for surrounding the animal's legs. The garment is made from double layered fabric. The cushioning qualities of the fabric are relied upon to provide protection for the animal.

U.S. Pat. No. 4,479,457 to Rotolo discloses a protective elbow pad for dogs. The pads are made from an outer layer of durable fabric, a foam central layer, and an inside layer of soft felt-like material. The elbow pad is adjustable in size and surrounds the immediate elbow area. None of the prior art device disclose a protective device for animals having a pocket that can receive padding or a hot or cold pack.

Most of the prior art devices are tightly surrounding the legs preventing the wound from breathing. Moreover, the prior art devices do not include an area to contain animal's paw; they do not provide protection when the animal's claws got injured. The prior art devices is not suitable for use if the animal is not cooperative and does to insert the leg into the sleeve.

Hence, it would be useful to have a device that allows the animal freedom to walk and maneuver while the wound is recovering. It would also be useful if the device allows the wound to breathe. It would also be useful if the sleeve of the device can wrap around animal's legs when the animal is not willingly or suitable to insert their legs into the sleeve of the device.

### SUMMARY OF THE INVENTION

It is an object of this invention to provide a pet leg protector that allows the animal to wear it loosely so that the wound can breathe.

It is an object of this invention to provide a pet leg protector that offers protection to animal's paws in addition to the legs. The paw area may be permanently or releasably attached to the leg area.

It is another object of this invention to provide a pet leg protector that has opening with zippers on the sleeve such that the sleeve can wrap around the animal's leg.

It is another object of this invention to provide a pet leg protector that uses material softer and more flexible for the area under the armpit area than the material used for leg and paw areas.

In an exemplary embodiment of the present invention, there is disclosed an apparatus for protecting wounds on the animal's leg or paw. The apparatus comprises a sleeve sized and shaped to loosely enclose the animal's leg and paw, as well as two straps. The sleeve includes a closed bottom paw area sized and shaped to contain the animal's paw, and a leg area connected to the top of the paw area sized and shaped to contain the animal's leg. The first strap connected to the left top of the sleeve includes an elastic material for easy adjustment and comfort; and a fastener located at an end of the first strap distal to the sleeve. The second strap connected to the right top of the sleeve includes an elastic material for easy adjustment and comfort, and a fastener located at an end of the second strap distal to the sleeve so as to complementarily match and engage the fastener of the first strap.

In one embodiment, the sleeve further includes an armpit patch made of flexible material located at the upper front or rear portion of the sleeve to adjust comfortably to the animal's armpit.

In one embodiment, the sleeve may have an opening and a fastener along its length such that the sleeve can wrap around the leg and closed by the fastener including one of a zipper, fabric fastener, snaps, and buttons.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a front view of the animal leg protector according to one embodiment of the present invention.

FIG. 2 is a side view of the leg protector showing the left side of the animal leg protector of FIG. 1.

FIG. 3 is a side view of the leg protector showing the right side of the animal leg protector of FIG. 1.

FIG. 4 is a side view of a dog wearing the animal leg protector on the front leg with two straps surrounding its chest and neck.

FIG. 5 is a side view of a dog wearing the animal leg protector on its hind leg with two straps surrounding its abdomen and back, and a third strap extending across its back to its chest.

FIG. 6 is a side view of a dog wearing the animal leg protector without paw area.

FIG. 7 is a side view of the animal leg protector with a removable paw area.

FIG. 8A is a front view of the animal leg protector having an opening with a zipper opened.

FIG. 8B is a front view of the animal leg protector having an opening with a zipper closed.

### DESCRIPTION OF THE PREFERRED EMBODIMENT

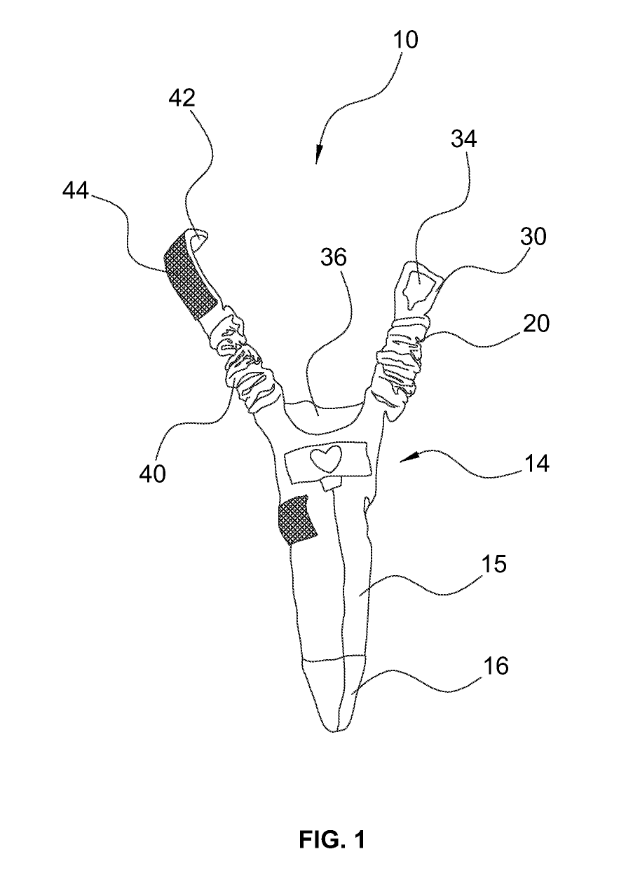

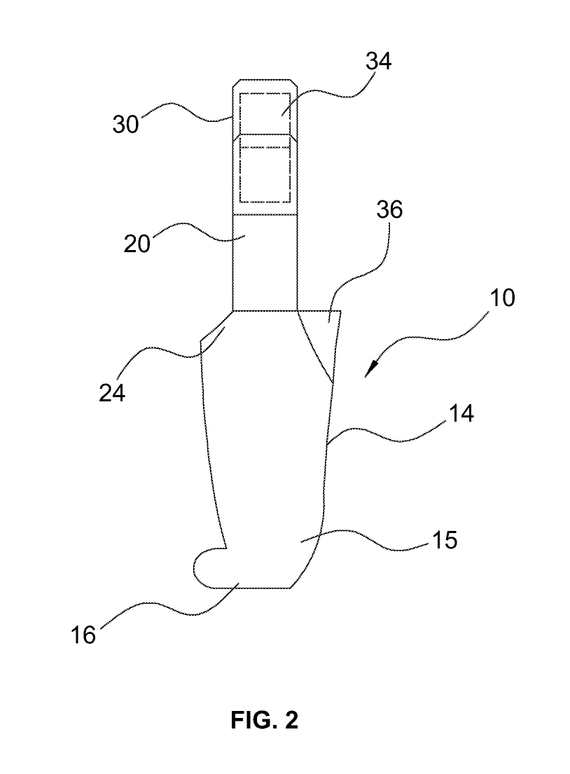

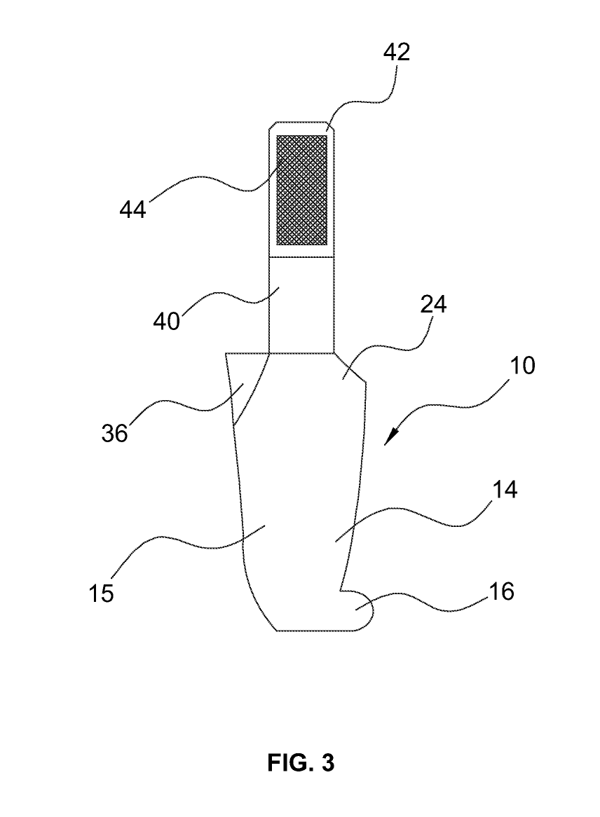

Referring to FIGS. 1-3, there is disclosed a front view, a left side view, and a right side view of the animal leg protector 10. As depicted in FIGS. 1-3, the animal leg protector 10 comprises a sleeve 14 which includes a leg are 15 sized and shaped to enclose the animal's leg loosely. The shape of the leg are 15 is of conically tapered shape. The sleeve 14 further includes a closed bottom paw are 16 which is sized and shaped to contain the animal's paw. The downward pressure of the animal's paw prevents the sleeve 14 from riding up the animal's leg and thus, holds the sleeve 14 in place. The sleeve 14 may be made of cotton, canvas, fleece, wool, plastic, mesh, rayon, nylon or other synthetic material, paper, or any other suitably flexible material known to those of ordinary skill in the art.

The animal leg protector 10 further comprises two straps. The first strap 20 is connected to the left side of the sleeve 14 at the top 24 of the leg are 15. The second strap 40 is connected to the right side of the sleeve 14 at the top 24 of the leg area 15. In one embodiment, the first strap 20 is sized and shaped to extend across the animal's abdomen and partially over the animal's back. The second strap 40 is sized and shaped to extend across partial of the animal's back. Both the straps 20, 40 may be made of elastic or other springing or flexible material to make it comfortably adjustable to the animal's shape and size, and to allow it to flex as the animal moves. Near or at the end 30 distal to the sleeve 14, the first strap 20 includes fastener 34 which may be a fabric fastener such as Velcro® hook and loop. The fastener 34 may also be a buckle, hook, or other fastener known to those of ordinary skill in the art. Near or at the end 42 of the second strap 40 distal to the sleeve, the strap includes a fastener 44 which is complementary to the fastener 34 on the first strap. The fastener 44 may include a fabric fastener such as Velcro® hook and loop, a buckle, hook, or other fastener known to those of ordinary skill in the art. The fasteners 34, 44 are properly positioned such that the first fastener 34 may engage the second fastener 44 to secure the two ends of the straps together. When engaged, the straps 20, 40 should be engaged at a sufficient length to engage the animal's paw and hold the leg protector 10 securely in place. The elastic in the straps 20, 40 allows the straps 20, 40 to extend and contract as the animal moves, so that the leg protector 10 remains securely in place.

The animal leg protector depicted in FIGS. 1-3 is designed for the animal's front leg. The sleeve 14 also comprises a patch 36 of fabric or patch at upper rear portion to allow the sleeve 14 to adjust comfortably to the animal's armpit area. This patch 36 may be of knitted fabric or other flexible material such that can flex as the animals leg works up and down inside the sleeve and helps to keep the animal's elbow inside the sleeve. The patch 36 may be located at upper front portion of the sleeve if the animal leg protector 10 is designed for the hind legs. Other elements of the animal leg protector for the front leg mentioned in the foregoing are same for the hind leg.

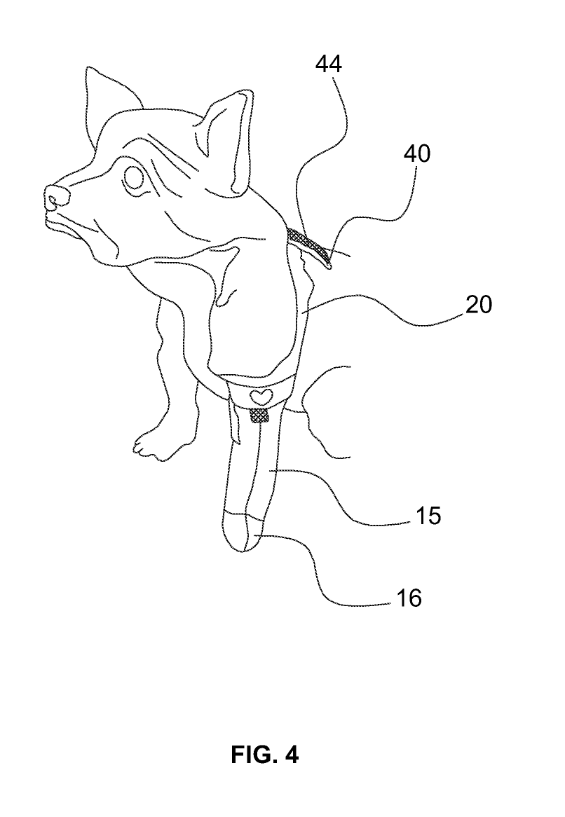

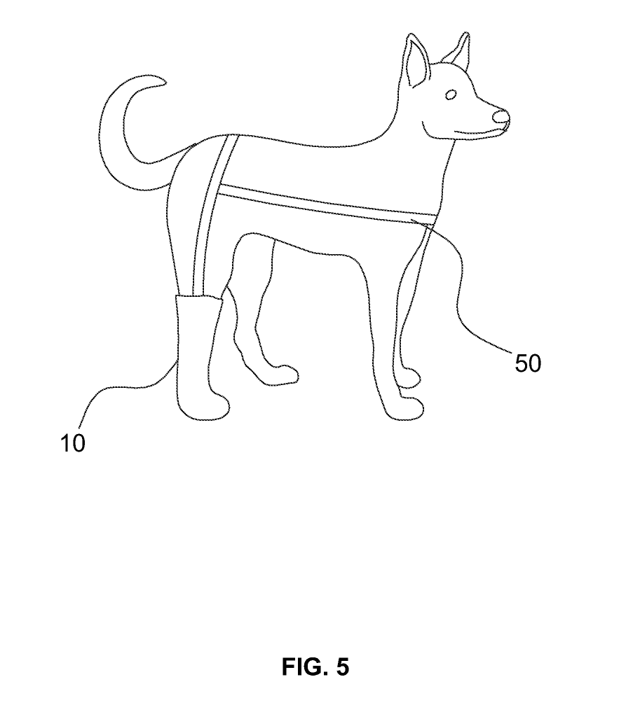

In an alternative embodiment, the straps 20, 40 may be sized and shaped to affix to the animal's neck. In another embodiment, the device may comprise one strap of sufficient length and elasticity that the strap may be extended to go over the animal's head for placement and removal of the device. In another embodiment, depicted in FIG. 4, the straps may be placed across the animal's back and under the animal's opposite armpit to maintain the device in position. In another embodiment, depicted in FIG. 5, the leg protector 10 comprises a third strap 50 that can be positioned across the animal's chest to hold the device in position. More straps may be used in another embodiment. This configuration would be especially useful in instances where the device is used on a hind leg of the animal.

In another embodiment, the leg protector 10 comprises a waterproof covering. This waterproof covering may be permanently affixed to the leg protector 10 as by sewing or any other means known to those of ordinary skill in the art. In an alternative embodiment the waterproof covering may be removably affixed to the leg protector by Velcro(R), snaps, buttons, hooks or by any other means known to those of ordinary skill in the art.

In another embodiment, the sleeve may also comprise a liner comprised of paper or other flexible material that may be inserted into the sleeve prior to use. The liner may be removed periodically and disposed of, whereupon it can be replaced by a fresh liner, thereby keeping the sleeve clean and sanitized.

In another embodiment, the entire leg protector is made of flexible disposable material such as paper and can be disposed of after each use. This disposable embodiment may comprise a removable waterproof cover for the paw area or for the paw and the leg area, such that the disposable leg protector may be disposed of after use and the waterproof cover may be reused.

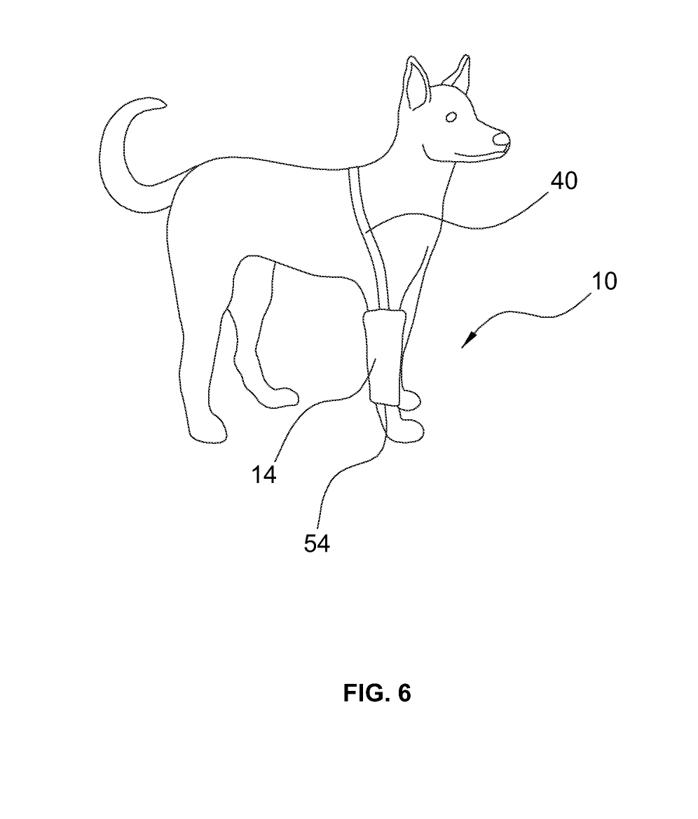

In another embodiment depicted in FIG. 6, the leg protector does not comprise an enclosure for the animal's paw. The leg protector comprises an elastic opening 54 at its lower end. This elastic opening expands and contracts to accommodate the area of the animal's ankle and to hold the sleeve securely in place to prevent it from traveling up the animal's leg.

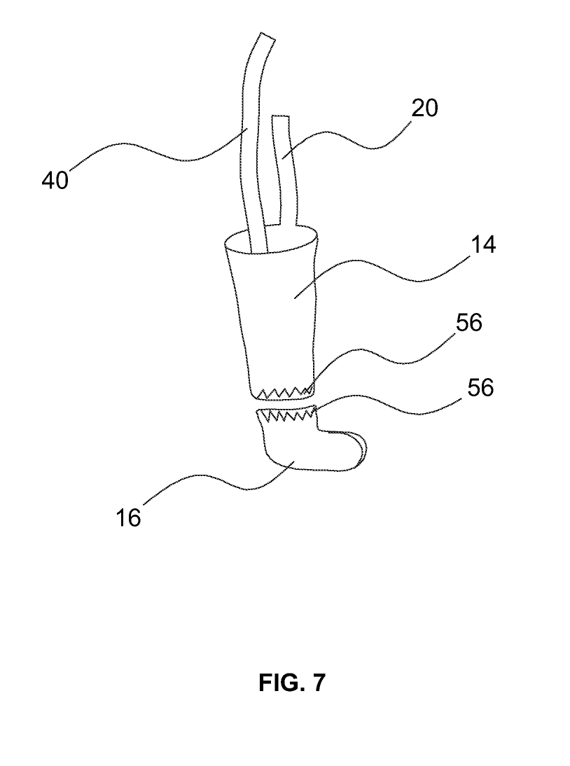

In another embodiment depicted in FIG. 7, the paw enclosure 16 is releasably attached to the leg protector 14 via fasteners 56. This can be accomplished by zipper, button, fabric fastener such as Velcro® hook and loop, or any other fastener known to those of ordinary skill in the art.

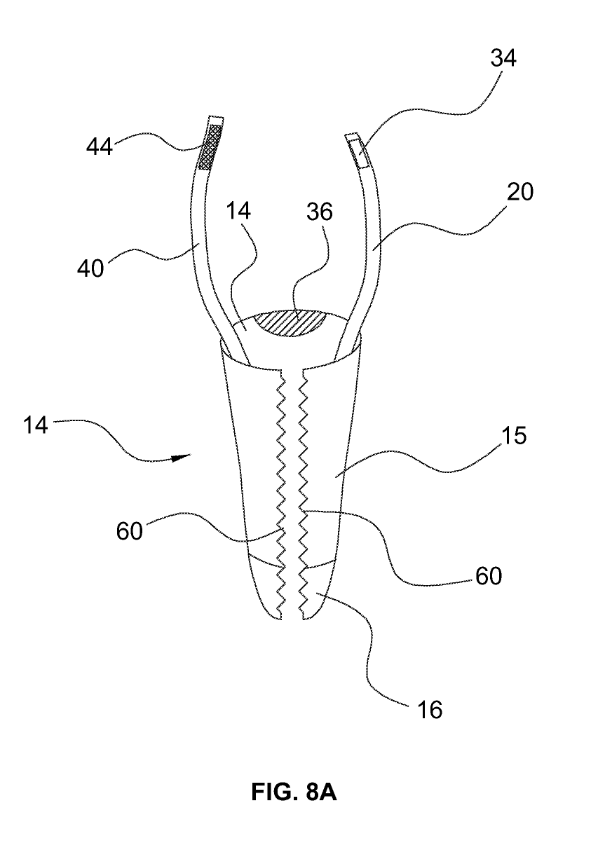

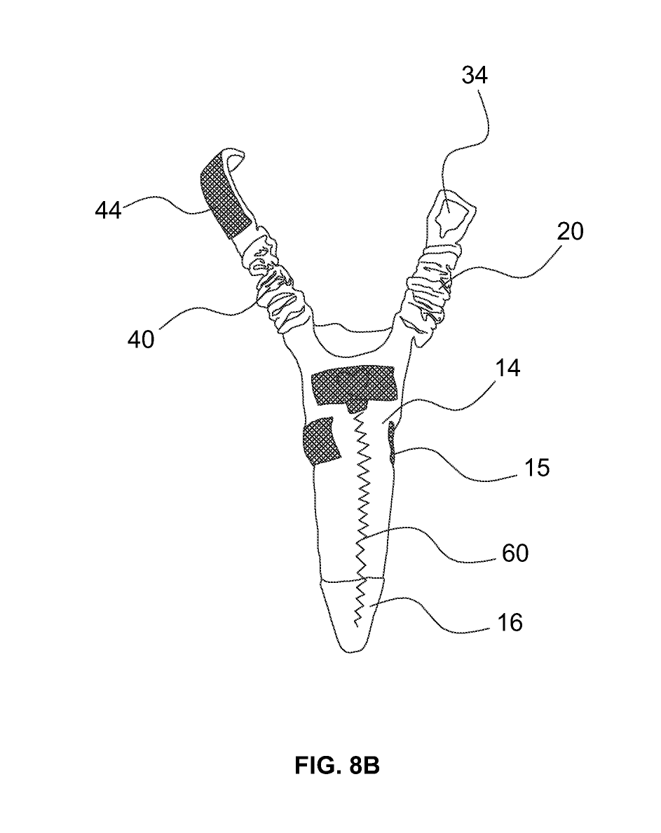

Referring to FIG. 8A and 8B, in another embodiment the sleeve may comprise an opening along its length. The opening assists in placing the protector on the animal's leg. This opening may be closed via fasteners 60 including a zipper, fabric fastener such as Velcro® hook and loop, snaps, buttons or any other means known to those of ordinary skill in the art.

In another embodiment of the present invention weights can be added to the outside of the leg protector to help with rehabilitation. Such weights could be removably attached to the device in varying degrees of heaviness to help build muscle or to return the leg to a normal functioning strength. Additionally, padding instead of weights could also be removably attached to add protection to the user of the device.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are entitled.

What is claimed is:

1. An apparatus for protecting wounds on the animal's leg or paw, the apparatus comprising:
a sleeve sized and shaped to loosely enclose the animal's leg and paw including:
a closed bottom paw area sized and shaped to contain the animal's paw;
a leg area connected to the top of the paw area sized and shaped to contain the animal's leg;
a first strap connected to the left top of the sleeve including:
an elastic material for easy adjustment and comfort; and
a fastener located at an end of the first strap distal to the sleeve; and
a second strap connected to the right top of the sleeve including:
an elastic material for easy adjustment and comfort; and
a fastener located at an end of the second strap distal to the sleeve so as to complementarily match and engage the fastener of the first strap.

2. The apparatus of claim 1, wherein the sleeve further includes an armpit patch made of flexible material located at the upper rear portion of the sleeve for the front leg or upper front portion of the sleeve for the hind leg to adjust comfortably to the animal's armpit.

3. The apparatus of claim 2, wherein the fastener is fabric fastener.

4. The apparatus of claim 3, wherein the sleeve is made of materials including one of cotton, canvas, fleece, wool, plastic, mesh, rayon, nylon, paper, and any combination thereof.

5. The apparatus of claim 4, wherein the sleeve has an opening and a fastener along its length such that the sleeve can wrap around the leg and closed by the fastener including one of a zipper, fabric fastener, snaps, and buttons.

6. The apparatus of claim 4, wherein the paw enclosure is removably attached to the bottom of the leg area via fasteners including one of zippers, buttons, and fabric fasteners.

7. The apparatus of claim 4, wherein the first strap is sized and shaped to extend over the animal's abdomen and partial of the back, and the second strap is sized and shaped to extend over partial of the animal's back and partially overlap the first strap such that the second strap's fastener engages the first strap's fastener whereby securing the two ends of the straps together on the animal's back.

8. The apparatus of claim 4, wherein the first and second straps are sized and shaped to go over the animal's chest and neck.

9. The apparatus of claim 4, wherein the first and second straps are sized and shaped to go over the animal's chest and back.

10. The apparatus of claim 4, further comprising a third strap to extend across the animal's chest to hold the device in place.

11. The apparatus of claim 4, further comprising a liner made of flexible material such that the liner can be removed periodically and disposed of and replaced by a fresh liner, thereby keeping the sleeve clean.

12. The apparatus of claim 4, wherein the paw area has waterproof covering.

13. An apparatus for protecting wounds on the animal's leg or paw, the apparatus comprising:
a sleeve sized and shaped to loosely enclose the animal's leg including:
a leg area sized and shaped to contain the animal's leg having an opening along its length for assisting in placing the protector on the animal's leg and the opening is closed via a fastener including one of a zipper, fabric fastener, snaps, and buttons;
an armpit patch made of flexible material located at the upper front or rear portion of the sleeve to adjust comfortably to the animal's armpit;
a first strap connected to the left top of the sleeve including:
an elastic material for easy adjustment and comfort; and
a fastener located at an end of the first strap distal to the sleeve; and
a second strap connected to the right top of the sleeve including:
an elastic material for easy adjustment and comfort; and
a fabric fastener located at an end of the second strap distal to the sleeve so as to complementarily match and engage the fastener of the first strap.

14. The apparatus of claim of 13 further comprises a paw enclosure sized and shaped to contain an animal's paw, wherein the paw enclosure is releasably attached to the sleeve via fasteners including one of zippers, buttons, and fabric fasteners.

15. The apparatus of claim 14, wherein the first strap is sized and shaped to extend over the animal's abdomen and partial of the back, and the second strap is sized and shaped to extend over partial of the animal's back and partially overlap the first strap such that the second strap's fastener engages the first strap's fastener whereby securing the two ends of the straps together on the animal's back.

16. The apparatus of claim 14, wherein the first and second straps are sized and shaped to go over the animal's chest and neck.

17. The apparatus of claim 14, wherein the first and second straps are sized and shaped to go over the animal's chest and back.

18. The apparatus of claim 14, further comprising a third strap to extend across the animal's chest to hold the device in place.

19. The apparatus of claim 14, further comprising a liner made of flexible material such that the liner can be removed periodically and disposed of and replaced by a fresh liner, thereby keeping the sleeve clean.

20. The apparatus of claim 14, wherein the fastener is fabric fastener.
